(19) United States
(12) Patent Application Publication
(10) Pub. No.: US 2015/0314092 A1
(43) Pub. Date: Nov. 5, 2015

(54) TRACHEAL TUBE WITH CONTROLLED-PRESSURE CUFF

(71) Applicant: Covidien LP, Mansfield, MA (US)

(72) Inventor: Gardner J. Kimm, Carlsbad, CA (US)

(73) Assignee: Covidien LP, Mansfield, MA (US)

(21) Appl. No.: 14/266,372

(22) Filed: Apr. 30, 2014

(51) Int. Cl. A61M 16/04

(52) U.S. Cl. CPC A61M 16/044; A61M 2205/3355; A61M 2205/07; A61M 2205/82; A61M 2205/502

(57) ABSTRACT

The present disclosure describes systems and methods that utilize a tracheal tube system with a piezoelectric cuff inflation system. The piezoelectric cuff inflator system may include an inlet, and outlet, and a piezoelectric element. In use the piezoelectric cuff inflator system may actuate the piezoelectric element to move a fluid from the inlet through the outlet suitable for inflating one or more cuffs included in the tracheal tube system. A circuitry may provide a frequency signal suitable for delivering a constant pressure to the cuff via the piezoelectric cuff inflation system.

### BACKGROUND

The present disclosure relates generally to medical devices and, more particularly, to tracheal tubes that include controlled-pressure cuffs.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tracheal tube (e.g., endotracheal, nasotracheal, or transtracheal device) may be used to control the flow of gases into the trachea of a patient. Often, a seal between the outside of the tube and the interior wall of the tracheal lumen is required, allowing for generation of positive intrathoracic pressure distal to the seal and prevention of ingress of solid or liquid matter into the lungs from proximal to the seal. The seal may be provided by using a cuff circumferentially disposed about a tube or lumen of the tracheal tube. The cuff may be inflated to a size suitable for abutting against the patient's airway, thus sealing the airway. It would be beneficial to provide for improved cuffs that more sealingly attach to the patient's airways.

### BRIEF DESCRIPTION OF THE DRAWINGS

Advantages of the disclosed techniques may become apparent upon reading the following detailed description and upon reference to the drawings in which:

FIG. 1 is a perspective view of a tracheal tube with a controlled-pressure cuff in accordance with embodiments of the present disclosure;

FIG. 2 is a cross sectional view of a piezoelectric pressure cuff inflator system in a power off or resting state;

FIG. 3 is a cross sectional view depicting an embodiment of the piezoelectric inflator of FIG. 2 when a piezoelectric element is actuated to provide a vacuum state;

FIG. 4 is 3 is a cross sectional view depicting an embodiment of the piezoelectric inflator of FIG. 2 when a piezoelectric element is actuated to provide a fluid discharge state;

FIG. 5 is a block diagram of an embodiment an inflation controller system of the piezoelectric inflator of FIG. 2;

FIG. 6 is a flow chart of an embodiment of a process for providing substantially constant pressure to a cuff via the piezoelectric pressure cuff inflator system of FIG. 2;

FIG. 7 is a side view depicting an embodiment of a parallel flow coupling of two piezoelectric pressure cuff inflator systems;

FIG. 8 is a side view depicting an embodiment of a serial flow coupling of two piezoelectric pressure cuff inflator systems; and

FIG. 9 is a side view depicting an embodiment of serial and parallel flow couplings of a plurality of piezoelectric pressure cuff inflator systems.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

During intubation, a tracheal tube is inserted into a patient's airway to isolate the lower airway and facilitate transfer of gases to and from the patient's lung. Certain airway products, such as endotracheal tubes and endobronchial tubes, are inserted through the patient's mouth and past upper respiratory anatomical features, e.g., the vocal cords. Other types of airway devices, such as tracheostomy tubes, may be inserted via a surgical incision to access the airway, i.e., a stoma. Regardless of the method of insertion, it is desirable for the outer diameter of the inserted tube to be sufficiently small to slide into the airway without damaging the device itself or causing undue discomfort for the patient. For airway devices that include inflatable balloon cuffs to seal the tracheal space, the profile of the cuff against the tube contributes to the overall outer diameter of the tracheal tube. A cuff is typically in a deflated state during insertion of a tracheal tube and is subsequently inflated after the tracheal tube is in place.

However, when inflated, the cuff may tend to lose shape over time, for example, because of small leaks in certain components of the cuff's inflation circuit (e.g., cuff inflation valve), leading to undesired sealing of the patient's airway. Accordingly, it would be beneficial to provide for a cuff that maintains cuff inflation pressure relatively constant, thus improving the seal. As described in detail below, embodiments of tracheal tubes having a controlled pressure cuff are provided herein. In particular, a piezoelectric inflation system is described, useful in coupling with a tracheal cuff and providing for enhanced control of cuff pressure. In one embodiment, the piezoelectric inflation system may continuously deliver an inflation gas (e.g., air) at a constant pressure with minimal energy expenditure and without specific controller attachments. That is, the piezoelectric inflation system may drive the inflation gas at a desired pressure without having to be communicatively coupled to a computing system, such as an inflation controller.

In another embodiment, the piezoelectric inflation system may include one or more check valves useful in minimizing or eliminating backflow of the inflation gas. In other embodiments, one or more piezoelectric inflation systems may be disposed in parallel and/or in series to provide for a desired inflation gas pressure and to minimize or eliminate backflow. Additionally, the piezoelectric inflator(s) may be turned on or off after a desired time, further minimizing energy usage of the pressure-controlled cuff system. By providing for a more energy efficient and pressure-controlled cuff, the techniques described herein may increase the efficiency of the seal created when the cuff is in use.

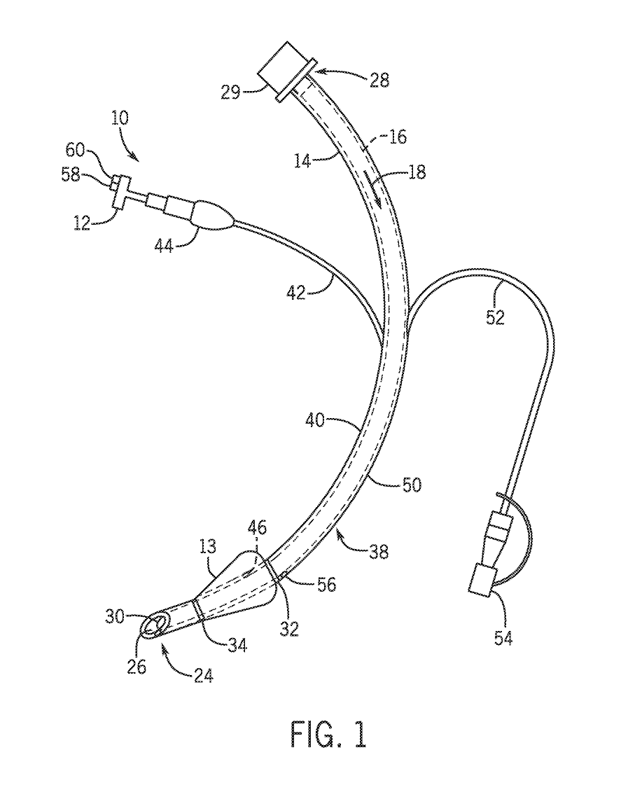

Turning now to the drawings, FIG. 1 is a perspective view of an exemplary tracheal tube 10 with a attached piezoelectric pressure cuff inflator system 12 suitable for controlling a pressure of an inflatable cuff 13, as described in more detail below. The tracheal tube 10 includes a central tubular body 14 that defines a ventilation lumen 16 that facilitates the transfer of gases to and from the lungs, e.g., as airflow into the lungs shown by arrow 18. The tracheal tube 10 is depicted with the inflatable cuff 13 disposed towards a distal end 24. The distal end 24 terminates in an opening 26. A proximal end 28 of the tracheal tube 10 may connect to upstream airway devices (e.g., a ventilator) via connector 29. A Murphy eye 30 may be located on the tubular body 14 opposite the opening 26, for example, to aid in preventing airway occlusion.

In operation, the cuff 13 is used to seal the tracheal space once inflated against the tracheal walls. The cuff 13 is typically affixed to the tubular body 14 via a proximal shoulder 32 and a distal shoulder 34. As noted, the present disclosure relates to controlling the pressure of the inflatable cuff 13. In certain embodiments, these techniques may be used in conjunction with multiple cuffs 13, oversized cuffs 13, undersized cuffs 13, and the like. The cuff 13 may be inflated via inflation lumen 40 terminating at its proximal end in an inflation tube 42 connected to an inflation pilot balloon and valve assembly 44. The inflation lumen 40 terminates at its distal end in notch 46. The piezoelectric pressure cuff inflator 12 may be coupled to the valve assembly 44, and used to provide for constant pressure when the cuff 13 is inflated. Additionally, it should be noted that the cuff 13 may be any suitable cuff, such as a tapered cuff, a non-tapered cuff, and so forth. The tracheal tube 10 may also include a suction lumen 50 that extends from a location on the tracheal tube 10 positioned outside the body and that terminates in a suction tube 52 and suctioning port 54 for suctioning secretions through opening 56.

The tracheal tube 10 and the cuff 13 are formed from materials having suitable mechanical properties (such as puncture resistance, pin hole resistance, tensile strength) and chemical properties (such as biocompatibility). In one embodiment, the walls of the cuff 13 are made of a polyurethane having suitable mechanical and chemical properties. An example of a suitable polyurethane is Dow Pellethane® 2363-80A. In another embodiment, the walls of the cuff 13 are made of a suitable polyvinyl chloride (PVC). In certain embodiments, the cuff 13 may be generally sized and shaped as a high volume, low pressure cuff that may be designed to be inflated to pressures between about 15 cm H₂O and 30 cm H₂O. The tracheal tube 10 may be coupled to a respiratory circuit (not shown) that allows one-way flow of expired gases away from the patient and one-way flow of inspired gases towards the patient. The respiratory circuit, including the tracheal tube 10, may include standard medical tubing made from suitable materials such as polyurethane, polyvinyl chloride (PVC), polyethylene teraphthalate (PETP), low-density polyethylene (LDPE), polypropylene, silicone, neoprene, polytetrafluoroethylene (PTFE), or polyisoprene. It is to be noted that the tracheal tube 10 may include multiple lumens 42, and the piezoelectric pressure cuff inflator 12 may be fluidly coupled to the multiple inflation lumens 42 via, for example, a Y-connector. Likewise, each of the multiple inflation lumens 42 may be coupled to individual piezoelectric pressure cuff inflators 12. Each piezoelectric pressure cuff inflators 12 may be powered by a power source 58 (e.g., battery) and controlled by an inflation controller system 60.

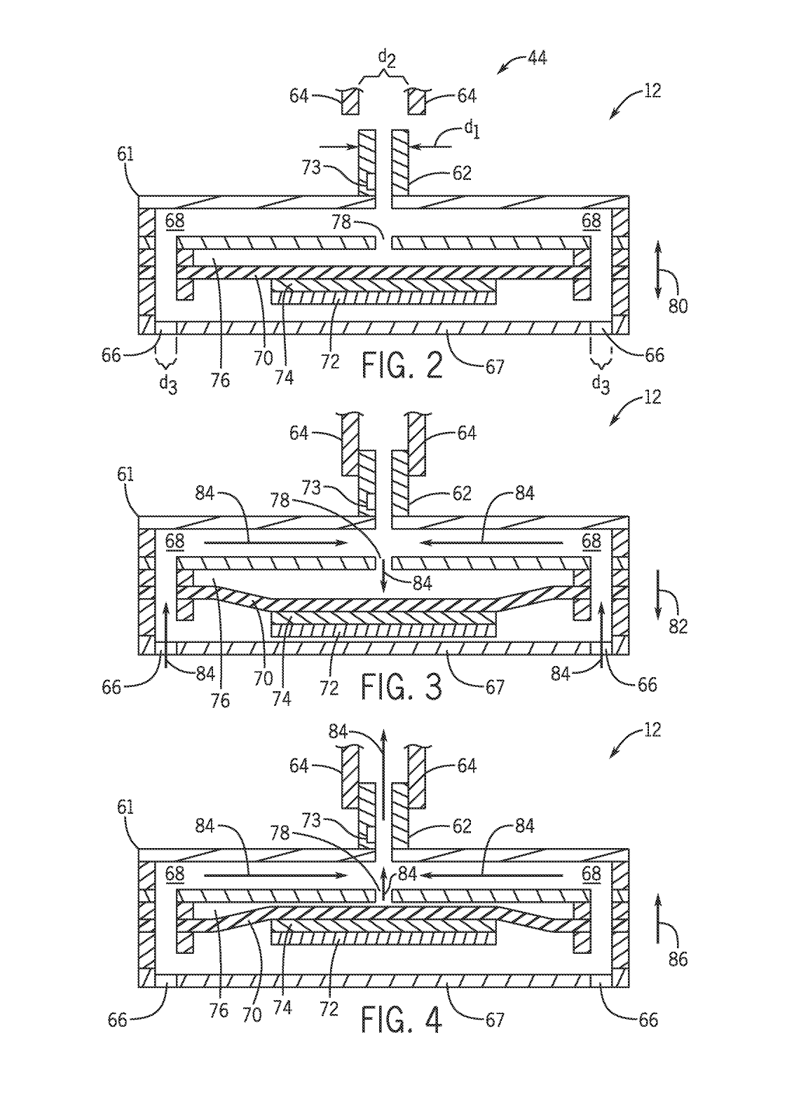

Traditionally, the cuff 13 may have been inflated using a standard syringe and the cuff's pressure may then have been determined using tactile feedback or measured directly via a manometer. Once inflated and the pressure derived, the cuff 13 may then slowly leak and may have to be re-inflated via the syringe, using up extra clinician or caregiver time. Likewise, movement of the patient, suctioning, and other activities, may lead to undesired changes (higher or lower pressures) in the cuff 13. The techniques described herein provide for attaching the piezoelectric pressure cuff inflator 12 after syringe inflation or as an alternative to syringe inflation, and the piezoelectric pressure cuff inflator 12 may then provide for a more constant pressure and use less energy when compared to other techniques (e.g., AC or DC powered air compressors), as described in more detail below with respect to FIG. 2.

FIG. 2 is a cross sectional view of the piezoelectric pressure cuff inflator 12 in a resting state (e.g., powered off). The piezoelectric inflator 12 includes a housing 61 having a fluid outlet 62, the outlet 62 having an outer diameter (OD) of d1. In use, the fluid outlet 62 may be disposed inside of a fluid inlet 64 included in the valve assembly 44 (shown in FIG. 1). Accordingly, the fluid inlet 64 may include an inner diameter (ID) d2 slightly larger than (or equal to) d1. An interference fit between the outlet 62 and the inlet 64 may then provide sufficient force to securely couple the piezoelectric inflator 12 to the valve assembly 44. The piezoelectric inflator 12 may be coupled to the fluid inlet 64 in a reversible fashion. That is, the end user may be capable of applying and removing the piezoelectric inflator 12. Alternatively, the piezoelectric inflator 12 may be permanently affixed to the intake line 42. In embodiments in which an inflator 12 is selected by a manufacturer or end user, a system 10 may include a kit or plurality of inflators 12 each with different attributes, including inflators 12 configured to provide for different inflation flow rates and pressures, e.g., a first inflator 12 having a first pressure or flow rate less than a second inflator 12. Likewise, the inflators 12 may be provided in different colors each color useful in denoting a particular pressure range and/or flow rate range. Similarly, the inflators 12 may be provided having a different diameter d1, where the diameter d1 is designed to only couple with inlets 64 fluidly coupled to certain types of cuffs, such as higher volume cuffs, lower volume cuffs, and the like. For example, the tracheal tube 10 may come in different sizes with correspondingly different sized cuffs 13. Accordingly, the inflator 12 may be selected based on the size of tracheal tube 10 used, the size of the cuff 13, and/or the number of cuffs 13 in use. Indeed, the techniques described herein may enable a more custom inflation based on selections of the inflator 12, and may additionally provide for an easier identification by the end user of which of the plurality of piezoelectric inflators 12 in the provided kit is more desirable to use in a given system 10 application.

The piezoelectric inflator 12 further includes a plurality of fluid inlets 66 with diameters d3 disposed in a wall 67 and fluidly coupled to the outlet 62 via a fluid passageway or conduit 68. The piezoelectric inflator 12 may additionally include a diaphragm 70 attached to a piezoelectric element 72 by an attachment member 74. The piezoelectric inflator 12 may further include a chamber 76 having an opening 78. In use, the controller system 60 may transmit an electric signal to the piezoelectric element 72 at certain desired frequencies, as described in more detail below, which will cause the piezoelectric element 72 to deform in accordance with the piezoelectric effect and thus move, for example, along the axis 80. As the signal is continuously applied, the axial movement of the piezoelectric element 72 may result in a pumping action suitable for moving fluid (e.g., air) from the inlets 66, through the passageway 68, and out through the outlet 62.

When the outlet 62 is coupled to the inlet 64 of the valve assembly 44, the fluid movement would then pressurize the cuff 13. Because the signal to the piezoelectric element 72 may be provided at the same frequency, an output flow rate, flow mass and/or pressure of the fluid exiting the outlet 62 may be constant, and may thus be controlled. Should patient movement or other adjustment cause the pressure cuff 13 to change in pressure, for example, by “pinching” the cuff outlet 13 to increase cuff pressure over the pressure provided by the piezoelectric inflator 12, the flow may reverse, and fluid from the pressure cuff 13 may flow out through the inlet 64 and into the outlet 62, traverse the passageway 68, and exit through the inlets 66. Accordingly, the piezoelectric inflator 12 may handle overpressure and then automatically re-inflate the cuff 13 with a desired pressure, thus keeping the pressure constant. For example, the piezoelectric inflator 12 may be operating continuously and inflating during the “pinching” event, and the backward flow of fluid entering the outlet 62 and exiting through the inlets 66 may have no adverse effect, with the piezoelectric inflator 12 reinflating the cuff 13 once the “pinching” event is over. In some embodiments, a pressure sensor 73 may be disposed on or in the outlet 62 to monitor the exhaust pressure. Other sensors may be disposed on or in the outlet 62, including flow sensors, temperature sensors, and the like. By inflating the cuff 13 at a desired pressure, the piezoelectric inflator 12 may improve a seal between the outside of the tube 10 and the interior wall of the patient's tracheal lumen, thus providing for improved respiratory support and delivery of medical fluids.

FIGS. 3 and 4 detail operations of the piezoelectric inflator 12 when a signal is applied to the piezoelectric inflator 12. Because FIGS. 3 and 4 include like elements to FIG. 2, the like elements are depicted using like numbers. More specifically, FIG. 3 is a cross sectional view depicting the piezoelectric inflator 12 when the piezoelectric element 72 is actuated to result in a vacuum state. As mentioned previously, communicating an electric signal, for example, via the control system 60, to the piezoelectric element 72 may result in the piezoelectric element 72 deforming in accordance to the piezoelectric effect. As the piezoelectric element 72 moves in a direction 82, the attached diaphragm 70 expands, resulting in a larger chamber 76 when compared to the chamber 76 at rest. Accordingly, a vacuum force may be provided, suitable for suctioning outside air 84 through the inlets 66, into the passageway 68, and then through the opening 78 into the chamber 76. Once the piezoelectric element 72 is near or has abutted the wall 67 of the housing 61, the piezoelectric element 72 may then move in a direction 86 as shown in FIG. 4.

As the piezoelectric element 72 moves in the direction 86, the attached diaphragm 70 may collapse into the chamber 76, thus reducing a volume of the chamber 76. During the collapse (e.g., discharge state), the piezoelectric element 72 provides for sufficient motive force through the diaphragm 70 suitable for the discharge of the air 84 through the outlet 62 and into the inlet 64 of the balloon and valve assembly 44. As can be appreciated, transitioning back and forth from the vacuum state (shown in FIG. 3) to the fluid discharge state (shown in FIG. 4) may provide for a continuous flow of the air 84 into the cuff 13. Additionally, because the flow pressure may be controlled, for example, via the controller system 60, a continuous flow of gas 84 may be delivered, resulting in a constant pressure for the cuff 13, as described in more detail below. The enclosure 61 and elements disposed therein, e.g., elements 66, 68, 70, 72, 74, and/or 76 may, in some embodiments, be available from Murata Manufacturing Corp. of Kyoto, Japan, as Microblower manufacturer part number MZB1001T02.

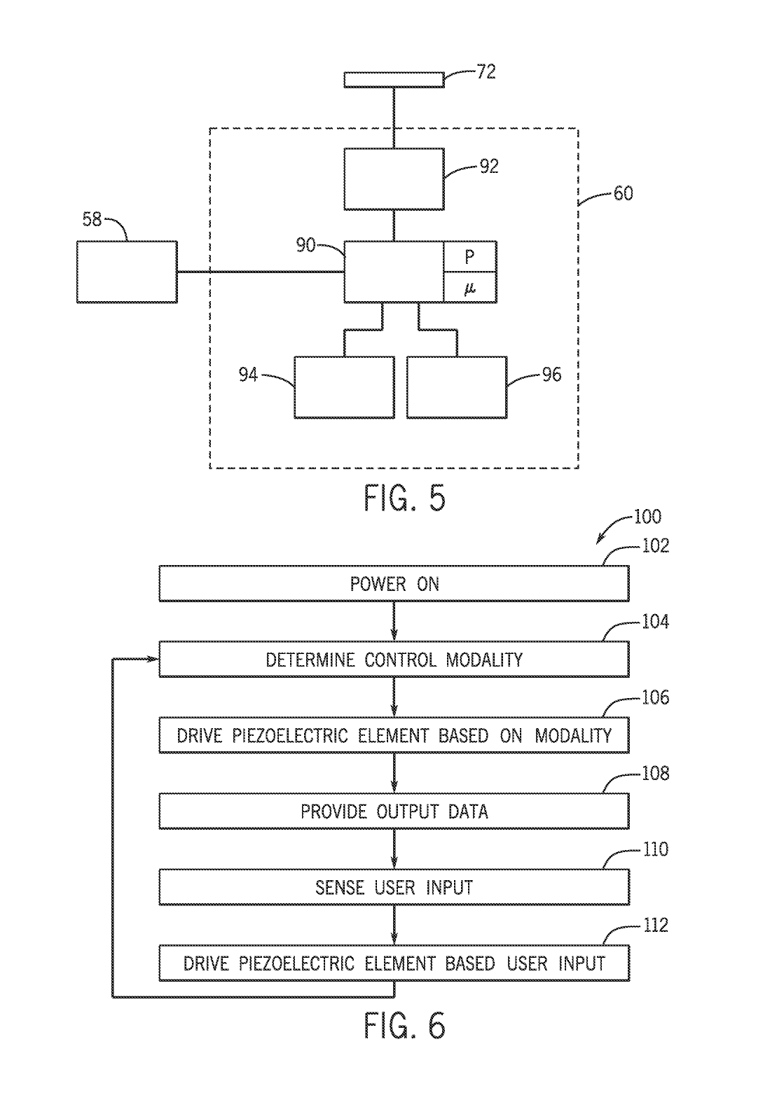

FIG. 5 is a block diagram of an embodiment of the inflation controller system 60 electrically coupled to the piezoelectric element 72. More specifically, the figure depicts a control circuitry 90 communicatively coupled to a signal generation circuitry 92. As depicted, the power supply 58 (e.g., battery) may deliver electric power to the control circuitry 90, which may then utilize the signal generation circuitry 92 to convert the electric power into a signal having a frequency useful in creating oscillations or movement of the piezoelectric element 72. The signal may then be delivered through electric conduit 94, such as a wire, a printed trace, and so on. The signal may be delivered at a driving frequency between 5-50 Hz, and a voltage peak-to-peak (Vp-p) of between 5-25 Vp-p. At 15 Vp-p, the air 84 pressure exiting the outlet 62 may be of approximately 1900 Pascal (Pa) when the outlet 62 has an ID of approximately 1.6 mm. If higher pressures are desired, the Vp-p and/or the frequency may be controlled. For example, lowering the Vp-p and/or frequency may result in lower pressures (e.g., lower than 1900 Pa), and increasing the Vp-p and/or frequency may result in higher pressures (e.g., higher than 1900 Pa). As mentioned earlier, a kit may include a plurality of piezoelectric inflators 12, and each piezoelectric inflator 12 may be provided at different inflation pressures and/or flow rates, for example, by adjusting the controller 60 so that each controller 60 delivers a different frequency signal from the other controllers 60, each frequency resulting in a given pressure and/or flow rate. Additionally, because pumping fluid via the piezoelectric effect may require less energy when compared to other pumping techniques, e.g., alternate current (AC) pumps, centrifugal pumps, solenoid actuating pumps, and the like, the techniques describe herein may provide for a more energy efficient and simpler device useful in maintaining the cuff 13 inflated to a desired pressure.

In the depicted embodiment, the circuitry 90 may include a processor 94 and a memory 96. The processor 94 may be a microprocessor configured to execute non-transitory computer code or instructions stored, for example, in the memory 96. In other embodiments, the processor 94 and the memory 96 may not be used, and the circuitry 90 may instead be a custom circuitry or programmable circuitry (e.g., ASIC, PAL, FPGA) and the like, configured to transform the electric power provided by the power supply 58 into the signal delivered to the piezoelectric element 72. Also depicted are user input circuitry 94 and user output circuitry 96. The user input circuitry 94 may include, for example, a power on and/or off switch, and one or more pressure adjustment switches. The switches may include Hall Effect switches, momentary switches, buttons, and the like. The pressure adjustment switch or switches may enable adjusting the pressure delivered via the piezoelectric inflator 12 by increasing or decreasing the delivered pressure. The user output circuitry 96 may include LEDs (e.g., OLEDs) and or other data visualization devices (e.g., display panel) that may display status information, including if the piezoelectric inflator 12 is on or off, battery 58 condition, and/or a pressure reading for the pressure exiting the outlet 62. The user output circuitry 96 may also include wireless circuitry (e.g., Bluetooth, Wi-Fi [IEEE 802.11x], Zigbee, near field communications [NFC], personal area networks [PAN]) useful in providing information (e.g., status information) wirelessly. The control system 60 may be used with a monitor, and, in certain embodiments, may be part of a ventilator that controls deliver of respiratory gases via the system 10. For example, the ventilator may issue commands to the control system 60 to adjust pressures, flow rates, to start and stop inflation, and so on. Likewise, the control system 60 may transmit signals (e.g., wireless signals) so that the monitor may display cuff pressures, flow rates, battery life, and the like.

FIG. 6 is a flowchart of an embodiment of a process 100 suitable for providing substantially constant pressure to the cuff 13 via the piezoelectric pressure cuff inflator system 12. The process 100 may be implemented as non-transitory computer readable instructions or code executable by the processor 94 and stored in the memory 96. The process 100 may be initiated by powering the circuitry 90 (block 102), thus commencing operations of the piezoelectric pressure cuff inflator system 12. The process 100 may then determine a control modality (block 104) to use during operations of the piezoelectric pressure cuff inflator system 12. In one control modality, the piezoelectric pressure cuff inflator system 12 may be operating continuously without interruption. Because of low power consumption, the piezoelectric pressure cuff inflator system 12 may operate for extending periods providing constant fluid flow at a desired pressure. In another control modality, the piezoelectric pressure cuff inflator system 12 may be operating cyclically, that is, providing fluid flow for a first desired time period, then turning off for a second desired time period, and then again providing fluid flow for the first desired time period. Cyclical operations may conserve power, and thus lead to even longer time between battery 58 changes. Control modalities may be user selected via the user input circuitry 94, or may be selected by the manufacturer. As noted previously, check valves may be used, additional to cyclical operations or alternative to cyclical operations.

Accordingly, the process 100 may then drive (block 106) the piezoelectric element 72 based on the control modality. In continuous flow modalities, the circuitry 90 may continuously deliver electric stimulation to the piezoelectric element 72 at a frequency and/or Vp-p suitable to create a desired fluid flow and fluid pressure through the outlet 62. In cyclical flow modalities, the circuitry 90 may alternate between delivering the electric stimulation for a desired time and stopping, thus further preserving electric power use. Cyclical modalities may also include varying cuff pressure based upon the airway pressure being created by the ventilator or other breathing device. For example, as airway pressure increases and decreases during the inspiratory and expiratory phases respectively, the cuff 13 pressure could also increase and decrease proportionally (or non-proportionally). Because the piezoelectric cuff inflator 12 is more closely coupled to the cuff (e.g., distance to cuff is shorter than when compared to using an external compressor attached to the cuff), the closer distance has advantages when used to vary pressure. For example, prior art cuff inflation systems that include a compressor located remotely typically require a longer pressure line. The longer pressure line may create a larger dead space, thus make it more difficult to rapidly change the pressure in the cuff. The piezoelectric cuff inflator system 12 does not include this larger dead space, and therefore can more quickly increase or decrease the pressure within the cuff.

In embodiments that include the sensor 73, both the continuous flow and the cyclical flow modalities may stop providing fluid through the outlet 62 if pressure readings are over a desired pressure range or set point. Alternatively, the controller 60 may modulate pressure and/or flow using closed loop control based upon a pressure sensor 73 or some other sensor (e.g., fluid flow sensor).

In one embodiment that includes the user output circuitry 96, the process 100 may provide output data (block 108). For example, status information may be displayed or wirelessly transmitted, including if the piezoelectric inflator 12 is on or off, battery 58 condition, flow rate, and/or a pressure reading for the pressure exiting the outlet 62. The process 100 may additionally sense user input (block 110). For example, the user input circuitry 94 may sense button presses, switch actuations, and/or commands transmitted wirelessly. Based on the sensed data, the process 100 may then drive the piezoelectric element 72 (block 112). For example, the user may request a different fluid flow rate and/or pressure, a different cycle time (for cyclical flow modalities), and/or may request to turn off the fluid flow or turn off the system 12. Accordingly, a different electric signal may be transmitted to the piezoelectric element 72, or no signal may be transmitted. User commands may include a change in control modality (e.g., from continuous to cyclical or vice versa). Therefore, the process 100 may iterate to block 104 to determine the control modality and continue process execution.

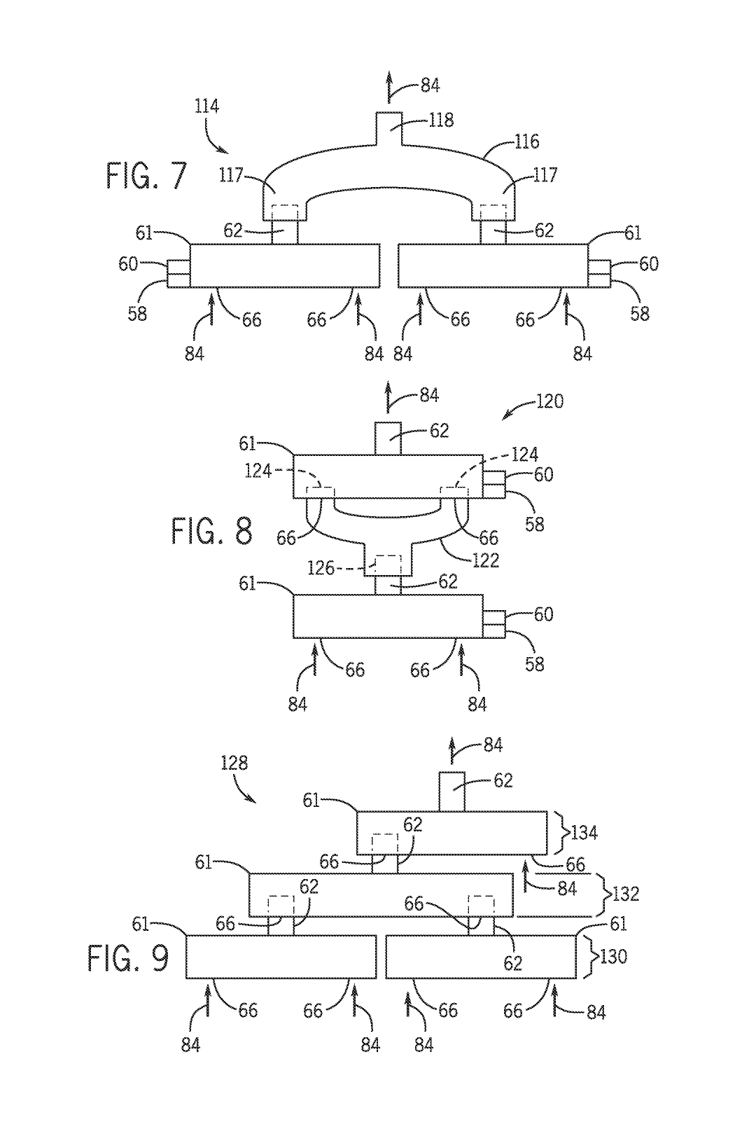

The techniques described herein may include attaching a plurality of the piezoelectric pressure cuff inflator systems 12 to the cuff 13 in a variety of couplings. For example, FIG. 7 is a side view depicting an embodiment of a parallel flow coupling 114 having two piezoelectric pressure cuff inflator systems 12. Because FIG. 7 includes like elements to FIGS. 2-4, the like elements are depicted using like numbers. In the depicted embodiment, an inverse Y-adaptor 116 may be used to fluidly couple the outlets 62 in parallel with multiple inlets 117 so as to provide for a single outlet 118. The outlet 118 may include the OD d1 suitable for insertion into the inlet 64 of the valve assembly 44 (shown in FIG. 1). The inlets 117 may include an ID d2 suitable for inserting the outlets 62. By providing for the parallel flow coupling 114, the fluid 84 may be delivered at higher (e.g., double) the flow rate when compared to using the single piezoelectric pressure cuff inflator systems 12 and also provide for fault tolerant operations. That is, should one of the piezoelectric pressure cuff inflator systems 12 stop operations due to an undesired condition, the second piezoelectric pressure cuff inflator system 12 may continue operations by providing for fluid 84 flow through the outlet 118.

FIG. 8 is a side view depicting an embodiment of a serial flow coupling 120 using two piezoelectric pressure cuff inflator systems 12 fluidly coupled in series via a Y-adaptor 122. Because FIG. 8 includes like elements to FIGS. 2-4, the like elements are depicted using like numbers. In the depicted coupling 120, the Y-adaptor 122 includes outlets 124 having an OD suitable for insertion into the inlets 66 of the piezoelectric pressure cuff inflator systems 12. The Y-adaptor 122 also includes an inlet 126 having an ID suitable for enabling the insertion of the outlet 62 inside of the inlet 126. Accordingly, inlets 66 of the lower piezoelectric pressure cuff inflator systems 12 may provide for air intake, the lower piezoelectric pressure cuff inflator systems 12 may then boost pressure and provide fluid flow into the upper piezoelectric pressure cuff inflator systems 12, and the upper piezoelectric pressure cuff inflator systems 12 may then provide additional fluid flow and pressure out through the upper outlet 62. By disposing systems 12 in series, the techniques described herein may provide for additional fluid flow, pressure, and/or fault tolerance in a more compact package. For example, should one of the two piezoelectric pressure cuff inflator systems 12 stop operations, the second piezoelectric pressure cuff inflator systems 12 would still operate and move the air 84 through the stopped piezoelectric pressure cuff inflator systems 12.

The piezoelectric pressure cuff inflator systems 12 may also be provided so as to enable “stackable” couplings, such as an embodiment of a coupling 128 depicted in FIG. 9. More specifically, FIG. 9 is a side view depicting various piezoelectric pressure cuff inflator systems 12 stacked on top of each other. Because FIG. 9 includes like elements to FIGS. 2-4, the like elements are depicted using like numbers. In the depicted embodiment, a first level 130 includes two piezoelectric pressure cuff inflator systems 12 disposed side-by-side. A second level 132 includes a single piezoelectric pressure cuff inflator system 12 disposed on top of the systems 12 of the first level. In the depicted embodiment, the inlets 66 of the system 12 in the second level 132 may have an ID approximately the same or slightly larger than the OD of the outlets 62 disposed in the first level 130. Accordingly, the first level 130 outlets 62 may be inserted into the inlets 66 of the second level 132 system 12. Additionally, a width of the housing 61 of system 12 that is shown disposed on the second level 132 may be wider than a width of the housing 61 of the systems 12 disposed on the first level 130 so as to enable side-by-side positioning of the systems 12 on the first level 130.

The piezoelectric pressure cuff inflator system 12 shown disposed on a third level 134 may also include inlets 66 having an ID approximately the same or slightly larger than an OD of the outlet 62 of the system 12 disposed on the second level 132. Accordingly, the third level 134 system 12 may be disposed on top of the second level 132 system 12. Accordingly, the piezoelectric pressure cuff inflator systems 12 may be “stacked” on top of one another to arrive at a variety of couplings, both parallel and serial. Similarly, the Y-adaptor 122 and inverse Y-adaptor 116 may be used with our without stacking embodiments to provide for a variety of serial and/or parallel couplings. By enabling a variety of couplings, such as couplings 114, 120, and 128, the techniques described herein may provide for more flexible and fault-tolerant systems 12 suitable for inflating cuffs at a variety of pressures.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheal tube system comprising:
a tracheal tube assembly comprising:
a cannula configured to be positioned in a patient airway;
a connector coupled to the proximal end of the cannula;
a cuff disposed about the cannula; and
a cuff inflation lumen comprising a first end fluidly coupled to the cuff and a second end configured to fluidly couple to a fluid-providing device; and
a piezoelectric cuff inflator system comprising an inlet, an outlet, and a piezoelectric element, wherein the outlet is configured to fluidly couple to the second end of the cuff inflation lumen for inflating the cuff during use via a fluid moved from the inlet through the outlet by actuation of the piezoelectric element.

2. The tracheal tube of claim 1, wherein the piezoelectric element is configured to convert an electric signal to an axial mechanical movement via a piezoelectric effect.

3. The tracheal tube of claim 2, wherein the piezoelectric cuff inflator system comprises a diaphragm coupled to the piezoelectric element and configured to flex axially based on the axial movement to increase or decrease a chamber space.

4. The tracheal tube of claim 3, wherein a chamber having the chamber space comprises a single opening fluidly coupled to the outlet, and wherein a first axial movement of the diaphragm moves fluid into the chamber through the opening, and a second axial movement of the diaphragm moves fluid out of the chamber through the opening.

5. The tracheal tube of claim 1, comprising a second piezoelectric cuff inflator system fluidly coupled to the piezoelectric cuff inflator system in parallel.

6. The tracheal tube of claim 1, comprising a second piezoelectric cuff inflator system fluidly coupled to the piezoelectric cuff inflator system in series.

7. The tracheal tube of claim 1, wherein the piezoelectric cuff inflator system comprises a controller configured to actuate the piezoelectric element by transmitting a signal to the piezoelectric element at a frequency range.

8. The tracheal tube of claim 7, wherein the piezoelectric cuff inflator system comprises a sensor communicatively coupled to the controller and configured to sense an outlet pressure, and wherein the controller is configured to adjust the signal based on the outlet pressure.

9. A tracheal tube system, comprising:
a piezoelectric cuff inflator system comprising:
an outlet having a first diameter sized to fluidly couple with a cuff inflation lumen end connector included in a tracheal tube;
an inlet having a second diameter and fluidly coupled to the outlet;
a piezoelectric element configured to move fluid inwardly into the inlet and outwardly through the outlet via the piezoelectric effect; and
a circuitry configured to provide a signal to the piezoelectric element to actuate the piezoelectric element via a piezoelectric effect.

10. The tracheal tube system of claim 9, comprising a chamber fluidly coupled to the inlet and to the outlet, wherein the piezoelectric element is configured to move the fluid from the inlet into the chamber and out of the chamber into the outlet.

11. The tracheal tube system of claim 10, wherein the chamber comprises a single opening.

12. The tracheal tube system of claim 10, wherein the chamber comprises a diaphragm coupled to the piezoelectric element, and wherein an axial movement of the piezoelectric element expands or contracts the diaphragm.

13. The tracheal tube system of claim 9, wherein the first diameter comprises an outer diameter (OD) sized to enable the insertion of the outlet into the inlet of a second piezoelectric system to create an interference fit between the outlet and the inlet of the second piezoelectric system.

14. The tracheal tube system of claim 13, wherein the second diameter comprises an inner diameter (ID) substantially equal to the OD.

15. The tracheal tube system of claim 9, comprising a controller including the circuitry configured to provide a signal to the piezoelectric element to actuate the piezoelectric element via a piezoelectric effect.

16. The tracheal tube system of claim 9, comprising a portable power source, wherein the circuitry is configured to receive power from the portable power source to provide the signal.

17. The tracheal tube system of claim 9, wherein the signal is provided depending on a user-selected mode.

18. A method of inflating a tracheal tube, comprising:
determining a desired inflation pressure for inflating a cuff included in a tracheal tube;
selecting a first piezoelectric cuff inflator system based on the desired inflation pressure; and
affixing a first piezoelectric cuff inflator system to an inflation lumen end connector, wherein the inflation lumen is fluidly coupled to the cuff, and wherein the piezoelectric cuff inflator system is configured to provide a first fluid flow into the inflation lumen at approximately the desired inflation pressure.

19. The method of claim 18, comprising affixing a second piezoelectric cuff inflator system to the first piezoelectric inflation system in series, in parallel, or a combination thereof, wherein the first and second piezoelectric cuff inflator systems are configured to provide a first fluid flow into the inflation lumen at approximately the desired inflation pressure.

20. The method of claim 19, wherein the affixing the second piezoelectric cuff inflator system to the first piezoelectric cuff inflator system in series comprises affixing a fluid adaptor between the first and the second piezoelectric cuff inflator systems, and wherein the affixing the second cuff piezoelectric inflation system to the first piezoelectric cuff inflator system in parallel comprises affixing an inverse fluid adaptor between the first and the second piezoelectric cuff inflator systems.

21. The method of claim 18, comprising first inflating the cuff with a syringe before affixing the first piezoelectric cuff inflator system.

22. The method of claim 18, comprising selecting the first piezoelectric cuff inflator system based on tracheal tube size.
